(19) United States
(12) Patent Application Publication
(10) Pub. No.: US 2013/0095468 A1
(43) Pub. Date: Apr. 18, 2013

(54) Method and Apparatus for Producing Ammonia and Nitrogen Fertilizer Using Nitrogen Fixing Bacteria

(71) Applicant: Baswood, Inc., Santa Barbara, CA (US)

(72) Inventor: Paul T. Baskis, Vero Beach, FL (US)

(73) Assignee: BASWOOD, INC., Santa Barbara, CA (US)

(21) Appl. No.: 13/649,549

(22) Filed: Oct. 11, 2012

Related U.S. Application Data
(60) Provisional application No. 61/546,360, filed on Oct. 12, 2011.

(51) Int. Cl. C12M 1/00

(52) U.S. Cl. CPC C12M 21/00
USPC 435/3; 435/289.1; 435/286.6; 435/168; 435/297.1

(57) ABSTRACT

The apparatus of the present invention includes a first nitrogen fixation tank open to the ambient air and nitrogen-fixing bacteria and a growth medium within the first nitrogen fixation tank downstream from the first nitrogen fixation tank for producing ammonium hydroxide from a combination of ambient air, water, and selected nutrients. The apparatus also includes a second evaporation tank to vaporize the ammonium hydroxide to form anhydrous ammonia.

The present invention relates to a method for producing ammonia and nitrogen fertilizer using nitrogen-fixing bacteria, including providing a first nitrogen fixation tank open to the ambient air, and nitrogen-fixing bactieria and a growth medium within the first nitrogen fixation tank for producing ammonium hydroxide from a combination of ambient air, water, and selected nutrients. The method also includes providing a second evaporation tank downstream from the first nitrogen fixation tank to vaporize the ammonium hydroxide to form anhydrous ammonia.

### CROSS-REFERENCE TO RELATED APPLICATIONS

This application claims the benefit of U.S. Provisional Application Ser. No. 61/546,360, filed Oct. 12, 2011, the contents of which are incorporated herein by reference.

### FEDERALLY SPONSORED RESEARCH OR DEVELOPMENT

N/A

### TECHNICAL FIELD

The invention relates generally to a method and apparatus for the production of ammonia and nitrogen for use as fertilizer.

### BACKGROUND OF THE INVENTION

Soils around the globe have become degraded due to intensive agricultural use. Farmers are currently experiencing lower crop yields despite their increased use of fertilizers and other chemicals.

One of the main problems with soils, particularly in the central portion of the United States, is a decrease in organic carbon present in the soil due to repeated cultivation and turning the soil over. These processes add oxygen to the soil, resulting in oxidation of the carbon in the soil, thus lowering the soil's productivity. This requires farmers to use more fertilizers.

One of the main advantages of the apparatus and method of the present invention is that it produces not only ammonia but also fertilizers and soil amendments that are rich in fixed carbon. This added fixed carbon increases the cation exchange capacity of the soil, as well as the soil's water holding capacity and air exchange, resulting in improved crop productivity. Additionally, the apparatus and method of the present invention also produces soil nutrients using less energy and with using little or no fossil fuels.

### SUMMARY OF THE INVENTION

The broad objective of the present invention is to provide an apparatus and method that uses carbon dioxide and nitrous oxides produced from combustion of methane either from biomass or from conventional natural gas sources to form acids that can be used to dissolve source rock into soluble nutrients. These nutrients are then used by nitrogen fixing bacteria to form ammonia that can be isolated and used as fertilizer for soil application. Some of the nitrogen fixed by the nitrogen fixing bacteria are used by these bacteria to grow and multiply. This nitrogen is collected in the biomass from the rapidly growing bacteria colony and is separated from the water and gasified to produce a solid fertilizer rich in readily available nitrogen phosphorous and potassium. The gasification of this biomass will produce more ammonia and also produces methane gas that can be used to power the generator that is producing the acids for dissolving the source rock.

In one embodiment, the apparatus of the present invention includes a first nitrogen fixation tank open to the ambient air and nitrogen-fixing bacteria and a growth medium within the first nitrogen fixation tank downstream from the first nitrogen fixation tank for producing ammonium hydroxide from a combination of ambient air, water, and selected nutrients. The apparatus also includes a second evaporation tank to vaporize the ammonium hydroxide to form anhydrous ammonia.

In another embodiment, the present invention relates to a method for producing ammonia and nitrogen fertilizer using nitrogen-fixing bacteria. The method includes the steps of providing a first nitrogen fixation tank open to the ambient air, and nitrogen-fixing bactieria and a growth medium within the first nitrogen fixation tank for producing ammonium hydroxide from a combination of ambient air, water, and selected nutrients. The method also includes the steps of providing a second evaporation tank downstream from the first nitrogen fixation tank to vaporize the ammonium hydroxide to form anhydrous ammonia.

In a further embodiment, the apparatus for producing ammonia and nitrogen fertilizer is provided. This apparatus includes a plurality of mineral processing tanks The mineral processing tanks each produce a mineral solution. It also includes a carbon source tank, and a nitrogen fixing tank containing nitrogen fixing bacteria and growth media for the bacteria. The nitrogen fixing tank receives the mineral solutions from the plurality of mineral tanks and carbon from the carbon source tank.

The apparatus of this embodiment further includes a settling tank for receiving water with dissolved ammonia from the nitrogen fixing tank, and separating nitrogen fixing bacteria and solids from the water. A first micron screen collects nitrogen fixing bacteria and solids from water received from the settling tank. A gas-liquid separator separates the dissolved ammonia from water received from the settling tank, and membrane filter collects remaining minerals in the water.

The apparatus also includes an aerobic digester for digesting residual organic compounds in water received from the gas-liquid separator, and a second micron screen for removing bacteria in water received from the aerobic digester. A gasifier heats solids received from the settling tank, the first and second micron screens, and minerals from the membrane filter sufficient to convert the solids and minerals to solid fertilizer. The gasifier also produces a mixture of methane gas and ammonia gas. A gas processor condenses ammonia gas received from the gasifier and the gas-liquid separator. A generator is provided for generating electric power from methane received from the gasifier, as is a scrubber for scrubbing exhaust gases from the generator.

### BRIEF DESCRIPTION OF THE DRAWINGS

To understand the present invention, it will now be described by way of example, with reference to the accompanying drawings in which:

FIG. 1 is a flow chart of the ammonia and nitrogen fertilizer producing process of an embodiment of the present invention;

FIG. 2 is a schematic of the ammonia and nitrogen fertilizer producing process of an embodiment of the present invention; and

FIG. 3 is a schematic drawing of the ammonia and nitrogen fertilizer producing process of a further embodiment of the present invention.

### DETAILED DESCRIPTION

While this invention is susceptible of embodiments in many different forms, there is shown in the drawings and will herein be described in detail preferred embodiments of the invention with the understanding that the present disclosure is to be considered as an exemplification of the principles of the invention and is not intended to limit the broad aspect of the invention to the embodiments illustrated.

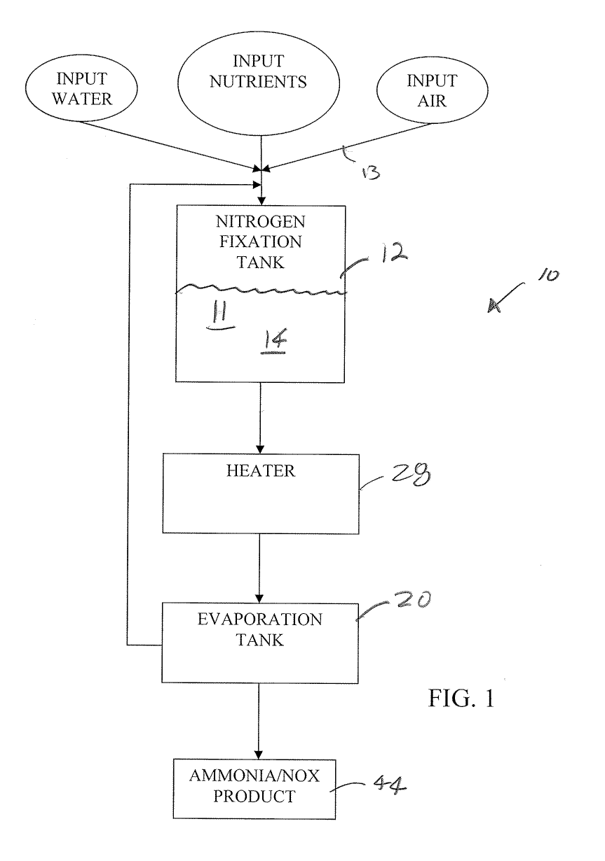

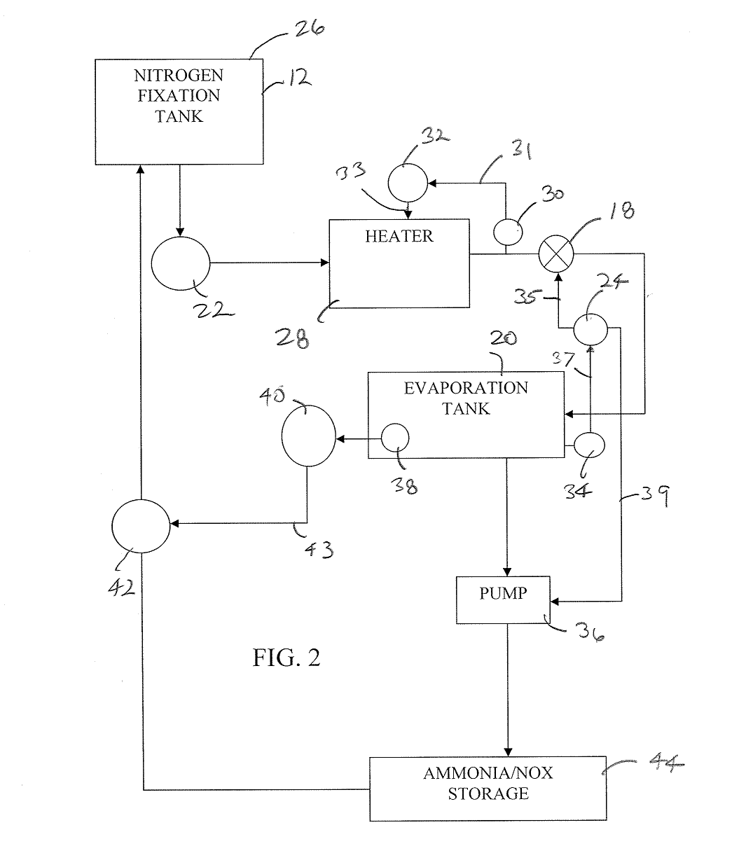

Referring to FIGS. 1 and 2, an apparatus 10 is shown for producing ammonia and nitrogen fertilizer using nitrogen fixing bacteria. Water, nutrients and air are inputted through pipes or conduits 13 or similar communication devices and introduced into a first nitrogen fixation tank 12. Suitable nutrients can include phosphorus, calcium, magnesium, iron, potassium, and carbon. The first tank 12 has within it a growth media 14, preferably of a plastic such as high density polyethylene (HDPE), or silica sand or quartzite sand. The HDPE media is advantageous where it is desirable to produce and collect the nitrogen fixing bacteria. Such bacteria do not readily affix themselves to the HDPE media, and can be collected using a settling tank and back flushing micron screens. This first tank 12 is filled with a mixture 11 of water, nutrients, and growth media to approximately one-half (½) of its capacity up to full capacity. The capacity of first tank 12 may be any suitable desired capacity. This mixture 11 is transferred via a conduit, pipeline, or similar device 16 through a valve 18 to a second evaporation tank 20 and through the growth media 14.

It should be noted this process is continuous. A transfer pump 22 is controlled by a controller 24. The first tank 12 provides the environment for the formation of ammonia from atmospheric nitrogen, or N₂, utilizing naturally occurring nitrogen fixing bacteria from the soil environment. Air is pulled into the tank 12 through a manhole 26 that allows the first tank 12 to be open to the surrounding atmosphere and the 78% concentration of nitrogen that is in the air.

After an appropriate aerobic fixing or fermentation time in first tank 12, typically on the order of eight (8) hours, the bacteria that are free living and capable of fixing nitrogen begin to flourish. As there is no nitrogen in the environment no other bacteria can survive. These bacteria that can fix nitrogen are saprophytes that live off of plant residue so they can use a broad range of simple carbon sources normally found from fermenting plant material in the soil environment. There are both aerobic and anaerobic free living nitrogen fixing bacteria. This means that the environment can vary in the amount of oxygen that is present and still produce excellent growth rates. These factors are such that growth of nitrogen fixing bacteria will be easy in a wide range of environments and a wide range of substrates as a carbon source making the economics most attractive. Examples of aerobic bacteria would be Azotobacter, Beijerinckia, and Azospirrillium. An anaerobic bacteria example would be Clostridium pasteurianum. The general chemical reaction for the fixation of nitrogen is identical for chemical and biological processes: N₂+3H₂→2NH₃. In an alternative embodiment, pure nitrogen can be obtained from a nitrogen generator, and an anaerobic (closed or fermentation) environment can grow the nitrogen fixing bacteria.

The transfer pump 22 moves the mixture 11, now containing ammonia in the form of ammonium hydroxide, through a heater 28. This occurs during the transfer of the mixture 11 into the second tank 20. The mixture 11 including ammonium hydroxide is heated to a desired temperature, which is near the boiling point of ammonium hydroxide, or approximately 97° F. The temperature of the transferred nutrients, water and ammonium hydroxide mixture 11 is controlled via a thermocouple 30 and heater controller 32. Thermocouple 30 is located downstream of heater 28. As a result, the temperature of the mixture 11 is measured after it passes through the heater 28. An output 31 of the thermocouple 30 is sent to the controller 32. The heater 28 is selectively turned on and off via an output 33 from the heater controller 32 to the heater 28.

The valve 18 permits the heated ammonium hydroxide, water and nutrients mixture 11 to pass into the second evaporation tank 20. Evaporation tank 20 contains no media therein, but preferably includes flat sloped plates with internal passages therein to allow the mixture 11 containing the ammonium hydroxide sufficient surface area to vaporize the ammonia into a gas, NH₃, normally referred to as anhydrous ammonia. Alternatively, the second evaporation tank 20 can be a heated stir tank. The valve 18 is preferably selectively operated via controller 24. Controller 24 operates the valve 18 and transfer pump 22 via an output 35 so that the valve 18 and transfer pump 22 are both activated simultaneously.

Controller 24 also receives data from a pressure sensor 34 measuring the pressure in the second tank 20 via output 37. The controller 24 through an output 39 controls a vacuum pump 36 keeping the second tank 20 at the desired set point, that being approximately −0.5 psig. Separate but linked controllers may also be used to control the transfer pump 22, valve 18, and vacuum pump 36. Whenever the vacuum of the second tank 20 is above the −0.5 psig, or alternative desired set point, the controller 24 activates the vacuum pump 36. When vacuum pump 36 is running it draws a vacuum in the second tank 20 and as the pressure lowers to −0.5 psig, or the desired set point, the valve 18, which is normally closed, is activated and opened by the controller 24. Simultaneously, the transfer pump 22 activates so as to transfer the mixture 11 through the heater 28 and into the second tank 20. This allows mixture 11 from first tank 12 that is heated to near the boiling point of ammonium hydroxide to pass through valve 18 and this begins decreasing the vacuum to above −0.5 psig, resulting in activation of transfer pump 22.

This cycle continues until the point where the level of water and nutrients 11 reaches the set point level as measured by a level sensor 38 in second evaporation tank 20. The level sensor 38 provides an output 41 to a second controller 40. Second controller 40 controls a recirculation pump 42. The controller 40 activates recirculation pump 42 reducing the level of contents in second tank 20 via output 43. The process of reducing the level in tank 20 also causes a reduction in vacuum of second tank 20 that pressure sensor 34 registers sending a signal to controller 24 via output 37 that again causes the activation of valve 18 and transfer pump 22. This provides a means of equalizing the vacuum in the second tank 20 and holds it at the set point that is desired.

To form very pure anhydrous ammonia may require some post processing steps but the formed product should be relatively high in ammonia of around 80%. Storage 44 is provided for collecting the ammonia product. It is stored as a product for sale. The liquid contents of second tank 20 are now sufficiently reduced in nitrogen content by the removal of the fixed ammonia and is transferred back to the first nitrogen fixing tank 12 and repeats the process.

In another embodiment, a digester can be provided for cleaning up the liquid contents of second tank 20 before its return to the first tank 12. The digester cleans the water and removes any small amounts of bacteria bodies and nitrogen that may have been converted into bacteria biomass. A final filtration of the liquid contents of second tank 20 using a membrane may also be used. Alternatively, the liquid contents of second tank 20 could be concentrated with other minerals that can also be used as a fertilizer product.

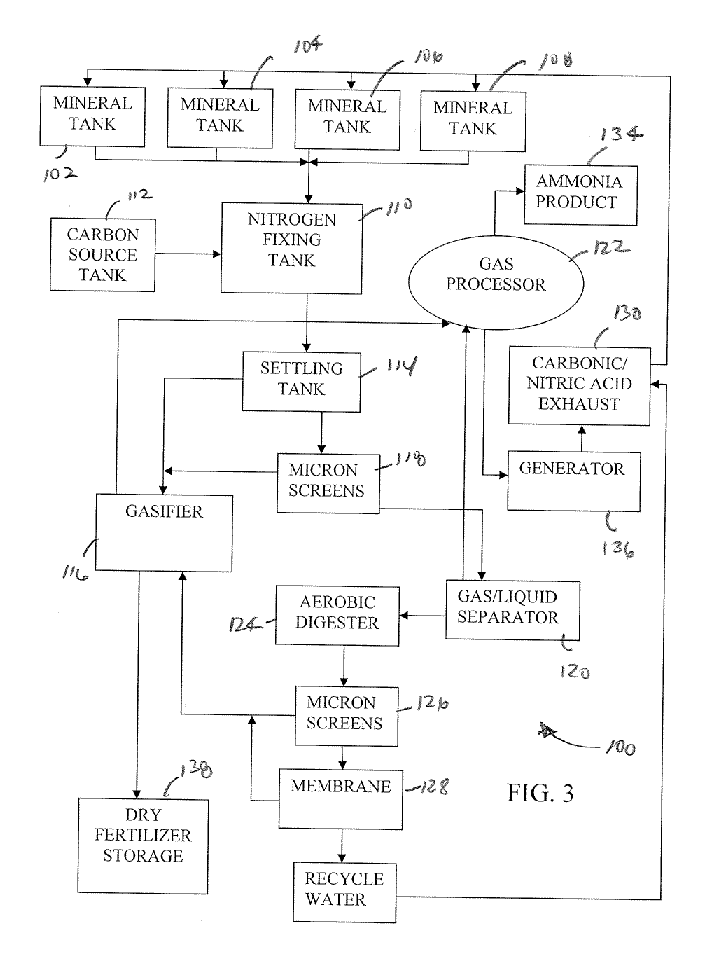

FIG. 3 is a schematic diagram of a further embodiment of the ammonia and nitrogen fertilizer producing apparatus and process 100 of the present invention. The apparatus and process 100 includes mineral processing tanks 102, 104, 106, and 108. Tank 102 is filled with apatite rock, and primarily supplies phosphorous to a nitrogen fixing tank 110. Tank 104 is filled with ground granite rock, and primarily supplies magnesium and potassium to the nitrogen fixing tank 110. Tank 106 is filled with limestone, and primarily supplies calcium to the nitrogen fixing tank 110. Tank 108 is filled with iron-cemented sandstone or other iron rich ore, and primarily supplies iron to the nitrogen fixing tank 110. The tanks 102 through 108 are preferably filled with their minerals from the top. The tanks supply the necessary minerals in a solution to the nitrogen fixing tank 110.

Tanks 102 through 108 each contain nitric acid and carbonic acid from a generator exhaust scrubber, to be described below. The acids are preferably pumped from the bottom to the top of the tanks The output from the tanks 102 through 108 is directed to the nitrogen fixing tank 110 by appropriate piping and pumps.

The nitrogen fixing tank 110 includes a selected growth media for growth of nitrogen fixing bacteria. Preferred growth media include high density polyethylene (HDPE) and quartzite filter sand. The HDPE media is advantageous where it is desirable to produce and collect the nitrogen fixing bacteria. Such bacteria do not readily affix themselves to the HDPE media, and can be collected using a settling tank and back flushing micron screens. A carbon source tank 112 contains a simple sugar such as glucose, which provides a simple carbon compound for the nitrogen fixing bacteria to use for growth and development.

In an embodiment, the nutrient concentration requirements obtained from tanks 102 through 108 and 112 for the nitrogen fixing bacteria in tank 110 are: 0.5 grams/liter magnesium sulfate, 0.25 grams/liter dipotassium phosphate, 0.25 grams/liter monopotassium phosphate, 0.5 grams/liter calcium chloride, 0.5 grams/liter iron (III) chloride, and 0.2 grams per liter glucose. In addition, at least one of the minerals would need to contain a sulfate. Thus, iron sulfate ore could be used rather than sandstone in tank 108. Sulfur is essentially ubiquitous, and most ores would contain a satisfactory amount of sulfur. Likewise, almost any sedimentary rock would contain adequate amounts of chloride.

A settling tank 114 receives water containing ammonia and free living nitrogen fixing bacteria from the nitrogen fixing tank 110. Settling tank 114 separates the bacteria and other solids from the water containing ammonia. The bacteria settling typically begins to occur within 60 to 180 seconds once in the settling tank 114. Any solids from the settling tank 114 are directed toward a gasifier 116. The water containing ammonia from the settling tank 114 are directed to a first micron screen 118. The first micron screen 118 is preferably capable of back flushing for cleaning and for collecting any remaining nitrogen fixing bacteria in the water. Solids from the first micron screen 118 are also directed toward the gasifier 116. Water from the first micron screen 118 is transferred to a gas-liquid separator 120.

The gas-liquid separator 120 separates the dissolved ammonia from the water containing ammonia. The water containing ammonia is heated to above the boiling point of ammonia, approximately 97 degrees Fahrenheit, while lowering the pressure above the water to a negative pressure. The resulting ammonia vapor is collected in a gas processor 122. The remaining water after the ammonia is extracted is transferred to an aerobic digester 124 for cleaning and reuse.

The aerobic digester 124 digests residual organic components in the water. The organic components result from nitrogen fixing bacteria growth. The organic components are unwanted byproducts as their development may shut down the enzyme system that allows for nitrogen fixation. Water from the digester 124 is directed to a second micron screen 126.

The second micron screen 126 cleans water from the digester 124 to remove any bacteria that may have grown in the aerobic digester 124. Solids collected by the second micron screen 126 are directed toward the gasifier 116. Water passing through the second micron screen 126 is directed to a membrane filter 128.

The membrane filter 128 is the final cleaning for the water before it is re-used. Any minerals left in the water to this point are collected in the back flush of the membrane filter 128 and directed to the gasifier 116. Water is directed to a scrubber 130.

Gas processor 122 cryogenically under medium to high pressure condenses the ammonia from gasifier 116. The condensed ammonia is then directed to ammonia product storage 134 and methane is directed to a generator 136. The generator 136 can be used to generate electric power to run pumps and other devices used to drive and to control the process being described. Ammonia from the gas-liquid separator 120 is also separated and sent to storage in the gas processor 122.

Carbon dioxide and nitrous oxides produced from the exhaust of generator 136 are scrubbed from the exhaust. The carbon dioxides and nitrous oxides form a weak acid composed of carbonic and nitric acids. These acids are directed back to mineral tanks 102 through 108 and the carbon source tank 112 to produce the liquid nutrients to feed the nitrogen fixing bacteria.

The gasifier 116 receives all solids from the settling tank 114, the first micron screen 118, the second micron screen 126, and the liquid minerals from the membrane filter 128. In another embodiment, the liquid minerals from the membrane filter 128 may be kept separate from the solids. These materials become blended in the gasifier 116 and are heated to approximately 1,000 degrees Fahrenheit, converting them into a high carbon solid fertilizer rich in nitrogen, potassium, and phosphorus. Dry fertilizer storage 138 receives processed material from the gasifier 116 for storage.

While the present invention has been described with respect to ammonia production, it may also be used to produce stand alone fertilizers and nutrients such as potassium carbonate and mono-ammonium phosphate. Other nutrient fertilizer/soil conditioners rich in calcium and/or magnesium may also be produced using the appropriate growth medium.

While the specific embodiments have been illustrated and described, numerous modifications come to mind without significantly departing from the spirit of the invention, and the scope of protection is only limited by the scope of the accompanying Claims.

What is claimed is:

1. An apparatus for producing ammonia comprising:
a first nitrogen fixation tank open to the ambient air;
a growth medium within the first nitrogen fixation tank for producing ammonium hydroxide from a combination of ambient air, water, and selected nutrients;
nitrogen-fixing bacteria contained within the first nitrogen fixation tank; and
a second evaporation tank downstream from the first nitrogen fixation tank to vaporize the ammonium hydroxide to form anhydrous ammonia.

2. The apparatus of claim 1 further comprising:
a vacuum pump for maintaining negative pressure in the second evaporation tank and evacuating anhydrous ammonia from the second evaporation tank for storage;
a heater for heating the water, nutrients and ammonium hydroxide from the first nitrogen fixation tank;
a pressure sensor for measuring pressure in the second evaporation tank;
a controller for controlling the vacuum pump responsive to a selected pressure measured by the pressure sensor, the controller controlling the transfer pump responsive to a selected pressure measured by the pressure sensor, and simultaneously controlling the transfer valve;
a thermocouple for measuring the temperature downstream of the heater;
a heater controller for controlling the heater responsive to a selected temperature measured by the thermocouple;
a level sensor for measuring a liquid level in the second evaporation tank; and
a second controller for controlling the recirculation pump responsive to a selected liquid level measured by the level sensor.

3. The apparatus of claim 1 wherein the growth medium is selected from the group consisting of silica sand, quartzite sand, and plastic.

4. The apparatus of claim 1 wherein the selected nutrients include at least one nutrient selected from the group consisting of magnesium sulfate, dipotassium phosphate, monopotassium phosphate, calcium chloride, iron (III) chloride, and glucose.

5. The apparatus of claim 1 wherein the nitrogen-fixing bacteria include at least one bacteria selected from the group consisting of Azotobacter, Beijerinckia, and Azospirrillium, and Clostridium pasteurianum.

6. The apparatus of claim 2 wherein the heater heats the water, nutrients and ammonium hydroxide from the first nitrogen fixation tank to a temperature of approximately the boiling point of ammonium hydroxide.

7. The apparatus of claim 1 wherein the ammonia concentration in the anhydrous ammonia is approximately 80%.

8. The apparatus of claim 1 further comprising an aerobic digester for removing bacteria from the liquid contents of the second evaporation tank before recirculation to the first nitrogen fixing tank.

9. The apparatus of claim 2 wherein selected pressure for controlling the vacuum pump is −0.5 psig.

10. A method of producing ammonia comprising the steps of:
providing a first nitrogen fixation tank open to the ambient air;
providing nitrogen fixing bacteria and a growth medium within the first nitrogen fixation tank for producing ammonium hydroxide from a combination of ambient air, water, and selected nutrients; and
providing a second evaporation tank downstream from the first nitrogen fixation tank to vaporize the ammonium hydroxide to form anhydrous ammonia.

11. The method of claim 10 further comprising:
maintaining negative pressure in the second evaporation tank and evacuating anhydrous ammonia from the second evaporation tank for storage; and
recirculating liquid contents of the second evaporation tank to the first nitrogen fixation tank.

12. The method of claim 10 wherein the growth medium is selected from the group consisting of silica sand, quartzite sand, and plastic.

13. The method of claim 10 wherein the selected nutrients include at least one nutrient selected from the group consisting of magnesium sulfate, dipotassium phosphate, monopotassium phosphate, calcium chloride, iron (III) chloride, and glucose.

14. The method of claim 10 wherein the nitrogen-fixing bacteria include at least one bacteria selected from the group consisting of Azotobacter, Beijerinckia, Azospirrillium, and Clostridium pasteurianum.

15. The method of claim 10 wherein a heater heats the water, nutrients and ammonium hydroxide from the first nitrogen fixation tank to a temperature of approximately the boiling point of ammonium hydroxide.

16. The method of claim 10 wherein the ammonia concentration in the anhydrous ammonia is approximately 80%.

17. The method of claim 10 further comprising providing an aerobic digester for removing bacteria from the liquid contents of the second evaporation tank before recirculation to the first nitrogen fixing tank.

18. The method of claim 10 further comprising the steps of:
measuring pressure in the second evaporation tank;
controlling the transfer of the water, nutrients and ammonium hydroxide from the first nitrogen fixation tank to the second evaporation tank responsive to a selected pressure in the second evaporation tank;
measuring the temperature downstream of the heater;
controlling the heating of the water, nutrients and ammonium hydroxide from the first nitrogen fixation tank responsive to a selected temperature measured downstream of the heater;
measuring a liquid level in the second evaporation tank; and
controlling the recirculation pump responsive to a selected liquid level measured in the second evaporation tank.

19. The method of claim 18 wherein selected pressure in the second evaporation tank is −0.5 psig.

20. An apparatus for producing ammonia and nitrogen fertilizer comprising:
a plurality of mineral processing tanks, each mineral processing tank producing a mineral solution;
a carbon source tank;
a nitrogen fixing tank containing nitrogen fixing bacteria and growth media for the bacteria, and receiving the mineral solutions from the plurality of mineral tanks and carbon from the carbon source tank;
a settling tank for receiving water with dissolved ammonia from the nitrogen fixing tank, and separating nitrogen fixing bacteria and solids from the water;
a first micron screen for collecting nitrogen fixing bacteria and solids from water received from the settling tank;
a gas-liquid separator for separating the dissolved ammonia from water received from the settling tank;
a membrane filter for collecting remaining minerals in the water;
an aerobic digester for digesting residual organic compounds in water received from the gas-liquid separator;
a second micron screen for removing bacteria in water received from the aerobic digester;
a gasifier for heating solids received from the settling tank, the first and second micron screens, and minerals from the membrane filter sufficient to convert the solids and minerals to solid fertilizer, the gasifier also producing a mixture of methane gas and ammonia gas;
a gas processor for condensing ammonia gas received from the gasifier and the gas-liquid separator;
a generator for generating electric power from methane received from the gasifier; and
a scrubber for scrubbing exhaust gases from the generator.
